(19) United States
(12) Patent Application Publication
(10) Pub. No.: US 2003/0157023 A1
(43) Pub. Date: Aug. 21, 2003

(54) Microcapsules comprising functionalised polyalkylcyanoacrylates

(76) Inventors: Georg Roessling, Glienicke (DE); Andreas Briel, Berlin (DE); Nils Debus, Berlin (DE); Sabine Sydow, Berlin (DE); Birte Hofman, Wilhelmstorst (DE); Peter Hauff, Berlin (DE); Michael Reinhardt, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/221,727

(22) PCT Filed: Mar. 13, 2001
(86) PCT No.: PCT/EP01/02802

(30) Foreign Application Priority Data
Mar. 15, 2000 (DE) 100 13 850.0

(51) Int. Cl. A61B 8/00

(52) U.S. Cl. 424/9.52

(57) ABSTRACT

The invention relates to gas-filled microcapsules that consist of functionalized polyalkylcyanoacrylates that are produced by copolymerization of one or more alkylcyanoacrylates with a functional monomer and/or by partial side-chain hydrolysis of a polyalkylcyanoacrylate, as well as a process for the production of gas-filled microcapsules and their use for ultrasound diagnosis.

The objects of the invention are gas-filled microcapsules that contain functionalized polyalkylcyanoacrylate, especially for use in ultrasound diagnosis, as well as process for their production.

The application is based on the following definitions:

Microparticles: Generic term for all particles measuring between 500 nm and 500 μm, regardless of their structural design.

Microcapsules: All particles measuring between 500 nm and 500 μm with a nucleus-shell structure.

Wall material=shell material: Material of the microcapsule shell.

Nanoparticles: Generic term for all particles measuring less than 500 nm, regardless of their structural design.

Particles: Generic term for nanoparticles and microparticles.

Gas-filled microcapsules: Microcapsules with a gaseous core.

Homopolymers: Polymers made of a monomer.

Copolymer: Polymer made of various monomers.

Alkylcyanoacrylate: Alkylester of cyanoacrylic acid.

Polyalkylcyanoacrylate: Polymer made of one or more alkylcyanoacrylates essentially without free acid and alcohol groups.

Functional group: Molecule group that contains at least one polar, reactive atomic compound with an X—H group of atoms, with X=O, S and N.

Latent functional group: A functional group that is provided with a protective group, whereby the protective group can also protect several functional groups.

Functional monomer: Comonomer to alkylcyanoacrylates, which in addition to the polymerizing molecule group contains at least one free or latent functional group and with which a copolymer with free functional groups can be produced directly or after cleavage of the protective group.

Functionalized polyalkylcyanoacrylate: Polyalkylcyano-acrylate with free functional groups that can be produced by copolymerization of at least one alkylcyanoacrylate and at least one functional monomer or by partial side-chain hydrolysis of the esterified acidic function of polyalkylcyanoacrylates.

Functionalization: Production of functionalized polyalkylcyanoacrylates by copolymerization of at least one alkylcyanoacrylate and at least one functional monomer or by partial side-chain hydrolysis of the esterified acidic function of polyalkylcyanoacrylates.

Non-functionalized polyalkylcyanoacrylate: Polyalkyl-cyanoacrylate.

Gas-phase proportion Φ_{G}: Ratio of the gas volume to the total volume of the reaction batch=phase-volume proportion of gas in the reaction mixture.

Stirring is the mixing of a liquid with a liquid, solid or gaseous substance in such a way that the gas-phase proportion Φ_{G} is <1%.

Dispersing is the mixing of a liquid with a liquid, solid or gaseous substance in such a way that gas-phase proportion Φ_{G}>1%.

Dispersion is a colloidal (particle size <500 nm) or coarsely dispersed (particle size >500 nm) multi-phase system.

Primary dispersion is a colloidal dispersion that consists of polymer particles, produced by polymerization of one or more monomers.

Self-gassing is the introduction of gas into a liquid by the movement of the gas or by the production of a dynamic flow underpressure.

Flotation is the movement of gas-filled microcapsules directed against the acceleration force (acceleration due to gravity versus radial acceleration a) based on a difference in density between microcapsules and dispersing agents.

Floated material is the creamed layer of gas-filled microcapsules after flotation. As defined by the patent, the term polymer comprises both homopolymers and also copolymers, and the term polymerization comprises homopolymerization and copolymerization.

Alkylcyanoacrylates or polyalkylcyanoacrylates are used in a variety of ways in medicine and pharmaceutics.

The pharmaceutical agent Histoacryl® consists of, for example, butylcyanoacrylate and is used as tissue adhesive or vascular adhesive in surgery. After application, the monomer is polymerized and is able to seal tissue or vessels very quickly.

In addition, alkylcyanoacrylates are also proposed for depot formulation of active ingredients (Couvreur, P. et al. J. Pharm. Pharmacol. 31, 331-332 1979). In this case, the active ingredient or active ingredients is (are) embedded in a matrix that consists of the corresponding polymer. As a result, the speed and the site of the release of active ingredient can be modified and controlled.

In this case, alkylcyanoacrylates or polyalkylcyanoacrylates are suitable both for the production of active ingredient-containing implants measuring up to several centimeters and for the production of microparticles and nanoparticles measuring a few micrometers or nanometers.

The alkylcyanoacrylates or polyalkylcyanoacrylates have found a special application in the formulation of ultrasound contrast media.

As contrast media, substances that contain or release gases are generally used in medical ultrasound diagnosis, since with these substances, a more efficient density difference and thus impedance difference than between liquids or solids and blood can be produced.

The use of the terms “microparticles” and “microcapsules” is not uniform in the prior art. In the description below of the prior art, the definitions on which this application is based are used even if the terminology of the documents deviates therefrom.

In European Patents EP 0 398 935 and EP 0 458 745, gas-containing microcapsules are described as ultrasound contrast media that consist of synthetic, biodegradable polymer materials. Polyalkylcyanoacrylates and polylactides, i.a., are disclosed as wall materials. By process optimization, which is described in European Patent EP 0 644 777, the ultrasound activity of the gas-filled microcapsules that are described in EP 0 398 935 could be significantly improved. An increase of the ultrasound activity is achieved by the diameter of the air core having been enlarged in the case of constant particle diameter. Despite the smaller wall thickness that results therefrom, the particles nevertheless survive passing through the cardiopulmonary system. The shell of the disclosed gas-filled microcapsules is built up from polyalkylcyanoacrylates or polyesters of α-, β- or γ-hydroxycarboxylic acids.

The optimized production process for gas-filled microcapsules that consist of polyalkylcyanoacrylates is characterized in that the monomer is dispersed and polymerized in an acidic, gas-saturated, aqueous solution and in this case the build-up of microcapsules takes place directly. In this way, gas-filled microcapsules can be produced without organic solvents.

The gas-filled microcapsules of the prior art, whose shell material consists of polyalkylcyanoacrylates, have a number of drawbacks, however:

1. Polymers of alkylcyanoacrylates have no functional groups up to the terminal alcohol group, which are necessary for a direct covalent coupling of specifically binding molecules or the substances that influence kinetics.

2. Owing to the absence of functional groups and in comparison to functionalized polymers, polymers of alkylcyanoacrylates are similar in molecular weight and alkylcyanoacrylates are less water-soluble and less able to swell. In the case of an intravenous administration, the elimination of microcapsules from the blood circulation by the reticuloendothelial system of the liver depends strongly on the hydrophilicity of the particle surface, whereby hydrophobic surfaces accelerate the elimination. As a result, the diagnostic time window is limited.

3. In-vivo degradation is carried out by side-chain hydrolysis and depolymerization. In addition to the pH of the medium and the molecular weight of the polymer, the presence of functional groups is a more important parameter for the degradation in the blood and in the liver, whereby the degradation and the metabolization is generally carried out all the more quickly the higher the degree of functionalization.

4. Gas-filled microcapsules that consist of polyalkylcyanoacrylate have a limited stability against dilution, so that the ultrasound contrast medium dose has to be varied significantly when variation is done via the administration volume, but needs to be varied less when the variation is done via the ultrasound contrast medium concentration. Especially when done during an infusion, the option of diluting the contrast medium reduces the cost of administration.

The object of this invention was to provide gas-filled microcapsules for use in ultrasound diagnosis, which do not have the drawbacks of the prior art. A functionalization should open up the possibility of binding specifically binding molecules or the substances that influence kinetics to the polymer. In addition, a hydrophilization should be achieved to slow down the elimination of microcapsules from the blood circulation through the reticuloendothelial system of the liver and thus to enlarge the diagnostic time window. In addition, the degradation and the metabolization of the gas-filled microcapsules in the liver should be accelerated. Moreover, the ultrasound contrast media according to the invention should show a higher stability against dilution than the ultrasound contrast medium of the prior art, so that additional degrees of freedom in the variation of the dose to be administered and in the type of administration are produced.

The object of this invention is achieved by gas-filled microcapsules for use in ultrasound diagnosis that contain functionalized polyalkylcyanoacrylate. The functionalized polyalkylcyanoacrylate can be produced by copolymerization of one or more alkylcyanoacrylates, preferably butyl, ethyl and/or isopropyl cyanoacrylate, with a functional monomer, preferably cyanoacrylic acid, and/or by partial side-chain hydrolysis of a polyalkylcyanoacrylate, preferably polybutyl, polyethyl and/or polyisopropyl cyanoacrylate.

The production of gas-filled microcapsules, which contain functionalized polyalkylcyanoacrylate, can be carried out in various ways:

Process Variant I:

The first process variant is characterized by the following process steps:

(a) Mixing of the functional monomer with one or more alkylcyanoacrylates,

(b) In-situ copolymerization and build-up of microcapsules in acidic, aqueous solution under dispersing conditions in a process step.

Process Variant II:

The second process variant is characterized by the following process steps:

(a) Mixing of the functional monomer with one or more alkylcyanoacrylates,

(b) In-situ copolymerization in acidic, aqueous solution under stirring conditions, and

(c) Build-up of microcapsules under dispersing conditions separately from the copolymerization.

Process Variant III

The third process variant is characterized by the following process steps:

(a) In-situ polymerization of one or more alkylcyanoacrylates and build-up of microcapsules in acidic, aqueous solution under dispersing conditions in a process step,

(b) Implementation of partial side-chain hydrolysis by adding lye,

(c) Stopping of the reaction by the addition of acid.

Process Variant IV:

The fourth process variant is characterized by the following process steps:

(a) In-situ polymerization of one or more alkylcyanoacrylates in acidic, aqueous solution under stirring conditions,

(b) Build-up of microcapsules under dispersing conditions separately from the copolymerization,

(c) Implementation of partial side-chain hydrolysis by adding lye,

(d) Stopping the reaction by the addition of acid.

Process Variant V:

The fifth variant is characterized by the following process steps:

(a) In-situ polymerization of one or more alkylcyanoacrylates in acidic, aqueous solution under stirring conditions,

(b) Implementation of partial side-chain hydrolysis by adding lye in primary dispersion,

(c) Stopping the reaction by the addition of acid,

(d) Build-up of microcapsules under dispersing conditions optionally with renewed addition of one or more alkylcyanoacrylates.

Regardless of the process variant, one or more flotations with subsequent uptake of the floated material in a physiologically compatible medium optionally can be performed after the build-up of microcapsules has taken place.

In addition, even in the case of functionalization by copolymerization with a functional monomer that has already been performed, an additional functionalization optionally can be carried out by partial side-chain hydrolysis by adding lye and stopping the reaction by the addition of acid. In addition, process steps such as filtration, ultrafiltration and/or centrifuging for purification optionally can be implemented.

Regardless of the process variant, alkyl esters of cyanoacrylic acid are preferably used as monomers. Especially preferred are butyl, ethyl and isopropylcyanoacrylic acid.

As functional monomers, the following can be used:

Cyanoacrylic acid (H₂C═C(CN)—CO—OH), methacrylic acid (H₂C═C(CH₃)—CO—OH), methylenemalonic acid (H₂C═C(CO—OH)₂) and α-cyanosorbic acid (H₃C—CH═CH—CH═C(CN)—CO—OH) and their derivatives with the general formulas:

H₂C═C(CN)—CO—X—Z (cyanoacrylic acid derivatives),

H₂C═C(CH₃)—CO—X—Z (methacrylic acid derivatives),

H₂C═C(CO—X′—Z′)₂ (methylenemalonic acid derivatives) and

H₃C—CH═CH—CH═C(CN) —CO—X—Z (α-cyanosorbic acid derivatives) with

X=—O—, —NH— or —NR¹— and

Z=—H, —R²—NH₂, —R²—NH—R¹, —R²—SH, R²—OH, R²—HC(NH₂)—R¹

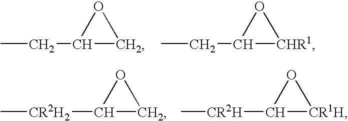

whereby R¹=linear or branched alkyl radical and R²=linear or branched alkylene radical with respectively 1 up to 20 carbon atoms, and whereby both X′ and Z′, in each case independently of one another, have the meaning that is indicated for X and Z.

Substituted styrenes (Y—C₆H₄—CH═CH₂) or methylstyrenes (Y—C₆H₄—C(CH₃)═CH₂) with

Y=—NH₂, —NR¹H, —OH, —SH, —R²—NH₂, —R²—NH—R¹, —R²—SH, R²—OH, —R²—HC (NH₂)—R¹

whereby R¹=linear or branched alkyl radical and R²=linear or branched alkylene radical with respectively 1 up to 20 carbon atoms.

Polymerizable emulsifiers (Surfmer), initiators with functionality (Inisurf) and chain-transfer agents with functionality (Transsurf)

Preferably used are:

Cyanoacrylic acid (H₂C═C(CN)—CO—OH) and Glycidylmethacrylate (

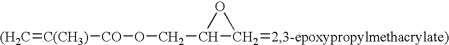

3-epoxypropylmethacrylate)

In this case, the functional monomer cyanoacrylic acid generates free carboxyl groups as functional groups with a polar, reactive O—H atomic group.

The functional monomer glycidylmethacrylate generates two free, vicinal alcohol groups (diol) with two polar, reactive O—H atomic groups. The alcohol groups are protected in glycidylmethacrylates in an epoxide group (latent functional groups) and are released by hydrolysis.

In process variants I and II, the functionalization is achieved by a copolymerization of the alkylcyanoacrylate with a functional monomer.

In process variants III to V, the functionalization is achieved by a subsequent treatment of polyalkylcyanoacrylate either in the primary dispersion or in the microcapsule suspension with lyes. In the alkaline medium, this leads to ester hydrolysis of the esterified acidic function in the side chain. Depending on the desired strength of the functionalization, such a reaction is carried out at a pH of 9-14 for about 15 minutes up to 5 hours at room temperature.

The reaction can be stopped with, for example, hydrochloric acid, by being adjusted to a pH below 7.

By variation of the pH and reaction time of the ester hydrolysis, a control of the degree of functionalization is possible. A pure surface functionalization is achieved if the reaction is carried out carefully.

The process step in process variants I and III, in which the polymerization and the build-up of microcapsules is carried out in one stage, is basically described in European Patents EP 0398935 and 0644777. Polymerization and build-up of microcapsules are carried out here in a process step under dispersing conditions. As dispersing tools, mainly rotor-stator-mixers are suitable, since the latter can produce a significant shear gradient and ensure a high introduction of gas by self-gassing.

The process step in process variants II, IV and V, in which the polymerization and the build-up of microcapsules is carried out in two stages, is the subject of a German Patent Application (Application Number: No. 19925311.0).

The invention that is described there relates to a multi-stage process for the production of gas-filled microcapsules, in which the process step of polymerization of the shell-shaping substance and the step of build-up of microcapsules take place separately. The microcapsules that are produced with the process according to the invention have a nucleus-shell structure and are distinguished by a defined size distribution.

The polymerization of the monomer is carried out in this case in acidic, aqueous solution under stirring conditions in such a way that the gas-phase proportion Φ_{G} is <1%. As an intermediate product of these process variants, a primary dispersion that consists of colloidal polymer particles is obtained. The diameter of the polymer latex particles that are produced for the encapsulation of gas lies in a range of 10 nm to 500 nm, preferably in a range of 30 nm to 150 nm, especially advantageously in a range of 60 nm to 120 nm.

The particle size of the colloidal polymer particles (characterizable by, for example, the average diameter and the polydispersity) and the molecular weight of the polymer (characterizable by, for example, the maximum value of the molar-mass distribution and the molar-mass distribution) can be influenced by, for example, the pH of the stirring medium, the surfactant concentration and the type of surfactant. In particular, the liquor bath ratio (quotient of the mass of surfactant and the mass of monomer) is an important parameter, by which the properties of the colloidal polymer particle can be controlled. The molecular weight of the polymer in this case influences the glass transition temperature of the polymer and thus its elasticity, a more important parameter for the acoustic properties of the gas-filled microcapsules that are produced from the colloidal polymer particles.

As stirring elements for the polymerization, basically all commonly used stirrers are considered, but especially those as they are used for the thorough mixing of low-viscous, water-like media (<10 mPas). These include, for example, propeller stirrers, vane stirrers, pitched-blade stirrers, MIG® stirrers and disk stirrers, etc.

In connection with the polymerization, a large proportion that is optionally produced during polymerization can be separated (e.g., by filtration) so that the latter no longer has a disruptive effect on the formation process of the microcapsules.

The formation of the gas-filled microcapsules is carried out in another step by structure-building aggregation of the colloidal polymer particles. The build-up of microcapsules from the polymer primary dispersion is carried out under dispersing conditions such that the gas phase proportion Φ_{G} is >1%, preferably greater than 10%. Formation of thrombi can be seen clearly. To this end, the primary dispersion must be stirred with a dispersing tool, so that the phase proportion of gas Φ_{G} in the reaction mixture is clearly above 1% in value and generally increases to more than 10%.

As dispersing tools in the production of gas-filled microcapsules in multi-stage processes, rotor-stator-mixers that can produce a high shear gradient are also suitable. In addition, they ensure a high introduction of gas.

The dimensions and the operating sizes of the dispersing tool(s) essentially determine the particle size distributions of the microcapsules; their sizing also depends on the size and cooling capacity of the unit.

A concrete process variant consists in performing the production of the primary dispersion in a continuous reactor, whereby to this end tube reactors with their tightly defined dwell-time behavior are more suitable than stirring vessel reactors.

By the suitable selection of polymerization parameters, the reactor geometry and the mean dwell time can be ensured in a simple way in a tube reactor, so that the polymerization at the end of the tube reactor is fully completed.

At the end of the tube reactor, a multi-stage rotor-stator system can be used for the build-up reaction of microcapsules, so that the entire process is performed in a single apparatus, and the two process steps, the production of a polymer dispersion and the build-up reaction of microcapsules nevertheless are decoupled from one another.

Another process variant calls for the use of a loop reactor, which consists of a continuous stirring vessel or optionally an intermittent stirring vessel with an outside loop, which contains a one- or multi-stage inline dispersing unit or a one- or multi-stage rotor-stator system, which in addition can produce the output for the outside loop.

In this case, the production of the primary dispersion is carried out either in the stirring vessel area under the moderate stirring conditions as well as in the closed loop or in the entire loop reactor when the loop is open, specifically under circulation conditions, which do not allow any self-gassing by correspondingly adjusted speed ranges. After the end of the reaction, the loop is opened to allow then the build-up reaction of microcapsules by the rotor-stator unit that is integrated in the loop. When the loop is open from the outset, the speed range of the rotor-stator unit increases accordingly.

Examples 1 and 2 provide process examples for the multi-stage build-up of microcapsules according to the above-mentioned German patent application.

Regardless of the process variant, the stirring or dispersing medium can contain one or more of the following surfactants:

Alkylarylpoly(oxyethylene)sulfate alkali salts, dextrans, poly(oxyethylenes), poly(oxypropylene)-poly(oxyethylene)-block polymers, ethoxylated fatty alcohols (cetomacrogols), ethoxylated fatty acids, alkylphenolpoly(oxyethylenes), copolymers of alkylphenolpoly(oxyethylene)(s) and aldehydes, partial fatty acid esters of sorbitan, partial fatty acid esters of poly(oxyethylene)sorbitan, fatty acid esters of poly(oxyethylene), fatty alcohol ethers of poly(oxyethylene), fatty acid esters of saccharose or macrogolglycerol ester, polyvinyl alcohols, poly(oxyethylene)hydroxy fatty acid esters, macrogols of multivalent alcohols, partial fatty acid esters.

One or more of the following surfactants are preferably used: ethoxylated nonylphenols, ethoxylated octylphenols, copolymers of aldehydes and octylphenolpoly(oxyethylene), ethoxylated glycerol-partial fatty acid esters, ethoxylated hydrogenated castor oil, poly(oxyethylene)-hydroxystearate, poly(oxypropylene)-poly(oxyethylene)-block polymers with a molar mass<20,000.

Especially preferred surfactants are:

Para-octylphenol-poly-(oxyethylene) with 9-10 ethoxy groups on average (=octoxynol 9,10), para-nonylphenol-poly(oxyethylene) with 30/40 ethoxy groups on average (=e.g., Emulan® 30, Emulan® 40), para-nonylphenol-poly(oxyethylene)-sulfate-Na salt with 28 ethoxy groups on average (=e.g., Disponil® AES), poly(oxyethylene)glycerol monostearate (e.g., Tagat® S), polyvinyl alcohol with a degree of polymerization of 600-700 and a degree of hydrolysis of 85%-90% (=e.g., Mowiol® 4-88), poly(oxyethylene)-660-hydroxystearic acid ester (=e.g., Solutol® HS 15), copolymer of formaldehyde and para-octylphenolpoly(oxyethylene) (=e.g., Triton® WR 1339), polyoxypropylene-polyoxyethylene-block polymers with a molar mass of about 12,000 and a polyoxyethylene proportion of about 70% (=e.g., Lutol® F127), ethoxylated cetylstearyl alcohol (=e.g., Cremophor® A25), ethoxylated castor oil (=e.g., Cremophor® EL).

Setting of the reaction speed of polymerization and the mean particle sizes resulting therefrom is carried out, i.a., in addition to the temperature by the pH, which can be set as a function of acid and concentration in a range of 1.0 to 4.5, for example by acids, such as hydrochloric acid, phosphoric acid and/or sulfuric acid.

Other values of influence on the reaction speed are the type and concentration of the surfactant and the type and concentration of additives.

The monomer is added at a concentration of 0.1 to 60%, preferably 0.1 to 10%, to acidic, aqueous solution.

The polymerization and the build-up of microcapsules are performed at temperatures of −10° C. to 60° C., preferably in a range of 0° C. to 50° C. and especially preferably between 5° C. and 35° C. The period of polymerization and the build-up of microcapsules lies between 2 minutes and 2 hours.

With the above-mentioned processes, in principle all gases in the microcapsules can be included if the reaction is carried out correctly. By way of example, there can be mentioned: air, nitrogen, oxygen, carbon dioxide, noble gases, nitrogen oxides, alkanes, alkenes, alkines, nitrous oxide, and perfluoro hydrocarbons.

The reaction batch can be worked up further.

The separation of gas-filled microcapsules from the reaction medium is advisable.

This can be done in a simple way with use of the density difference by flotation. The gas-filled microcapsules form a floated material, which can be separated easily from the reaction medium.

The floated material that is obtained can then be taken up with a physiologically compatible vehicle, in the simplest case water or physiological common salt solution.

The suspension can be administered directly. Dilution optionally is advisable.

The separation process can also be repeated one or more times. By specific setting of the flotation conditions, fractions with defined properties can be obtained.

The size and the size distribution of the microcapsules are determined by various process parameters, for example the shear gradient or the stirring period. The diameter of the gas-filled microcapsules lies in a range of 0.2-50 μm, in the case of parenteral agents preferably between 0.5 and 10 μm and especially preferably between 0.5 and 5 μm.

The suspensions are stable over a very long period, and the microcapsules do not aggregate.

The durability can nevertheless be improved by a subsequent freeze-drying optionally after the addition of polyvinylpyrrolidone, polyvinyl alcohol, gelatin, human serum albumin or another cryoprotector that is familiar to one skilled in the art.

The gas-filled microcapsules according to the invention can be used directly or optionally after activation for coupling specifically binding molecules or the substances that influence kinetics.

An activation of the functionalized polyalkylcyanoacrylate can optionally facilitate the coupling of specifically binding molecules and/or the substances that influence kinetics.

For example, activation with EDC (1-ethyl-3-(3-dimethylaminopropyl)-carbodiimide hydrochloride) can be carried out, by which an o-acylurea group is introduced in polymer-position as a group that can be coupled.

In this case, the binding of the molecule that is to be bound is preferably carried out by amine groups. To this end, the molecule to be bound optionally can be aminated (example: amine-terminated polyethylene glycol).

As specifically binding molecules, antibodies, preferably anti-EDB-FN-antibodies, anti-endostatin antibodies, anti-CollXVIII antibodies, anti-CM201 antibodies, anti-L-selectin-ligand antibodies, such as anti-PNAd antibodies (MECA79 antibodies), anti-CD105 antibodies, anti-ICAM1 antibodies or endogenic ligands, preferably L-selectin and especially preferably chimera L-selectin, can be used.

As substances that influence kinetics, synthetic polymers, preferably polyethylene glycol (PEG), proteins, preferably human serum albumin and/or saccharides, preferably dextran, can be used.

The specifically binding molecules or the substances that influence kinetics can either be coupled directly to the functional groups of the functionalized polyalkylcyanoacrylate via a spacer, for example protein G, or biotinylated via a streptavidin-biotin coupling to the gas-filled microcapsules.

The functional groups of the functionalized polyalkylcyanoacrylates can optionally be activated before the coupling reaction.

If no direct coupling is carried out, the spacers or the streptavidin are bonded in a first process step via the functional groups of the functionalized polyalkylcyanoacrylate to the gas-filled microcapsules. The specifically binding molecules or the substances that influence kinetics are then coupled to the spacer in the second process step or coupled to streptavidin in biotinylated form. Also in this case, the functional groups of the functionalized polyalkylcyanoacrylate optionally can be activated before the coupling reaction.

### EXAMPLE 1

### Non-Functionalized Gas-Filled Microcapsules

Multistage Process According to German Patent Application No. 19925311.0

(a) Production of the Primary Dispersion

For injection purposes, 500 ml of water is loaded into a 1 l glass reactor with a diameter to height ratio of 0.5, and a pH of 1.5 is set by adding 1N hydrochloric acid and a reactor temperature of 290.5 K is set. While being stirred with a propeller stirrer, 5.0 g of octoxynol is added and stirred until the octoxynol is completely dissolved. Then, 7 g of cyanoacrylic acid butyl ester is added in drops under the same stirring conditions over a period of 15 minutes, and it is stirred for another 2 hours.

(b) Production of the Microcapsule Suspension

The primary dispersion is dispersed for 2 hours with an Ultraturrax (e.g., IKA, T25 type) at high shear gradients (idle speed of the Ultraturrax about 20,500 min⁻¹). By the dispersing, a self-gassing of the process medium is carried out with the result of a strong formation of foam. After the end of the reaction, a creaming layer of gas-filled microcapsules is formed. For injection purposes, the floated material is separated from the reaction medium and taken up with 375 ml of water. The suspension that is thus obtained contains microcapsules in the range of 0.5-10 μm (laser diffractometer of the Malvern Instruments Company, MastersizerS type).

(c) Freeze-Drying

Then, 40 g of polyvinylpyrrolidone is dissolved in the batch, the suspension is formulated at 5 g and freeze-dried.

(d) Particle Size of the Nanoparticles in the Primary Dispersion

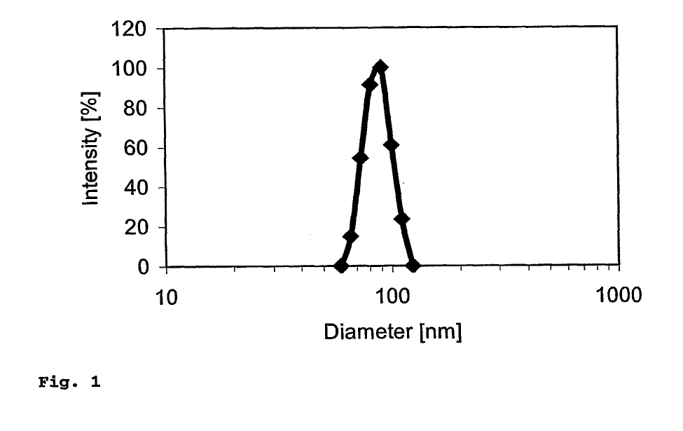

The primary dispersion that is obtained according to (a) is measured by means of dynamic light scattering (device: Nicomp Submicron Particle Sizer). FIG. 1 shows the measured size distribution of the nanoparticles. The mean diameter of the size distribution of 83 nm is intensity-weighted with a polydispersity index of about 25%.

### EXAMPLE 2

### Non-Functionalized Gas-Filled Microcapsules

Multistage Process According to German Patent Application No. 19925311.0

(a) Production of the Primary Dispersion:

1 l of an aqueous solution of 1% octoxynol at a pH of 2.5 is introduced into a 2 1 glass reactor with a diameter to height ratio of about 0.5 and an outside loop with a one-stage rotor-stator-mixing unit. 14 g of cyanoacrylic acid butyl ester is added in drops over 5 minutes and stirred for 30 minutes to be introduced without air into the reaction mixture.

(b) Production of the Microcapsule Suspension

For the production of the microcapsule suspension, the outside loop is attached to the circuit for 60 minutes, and the primary dispersion is dispersed. The stirrer in the glass reactor is set in such a way that a self-gassing of the reaction mixture is carried out. After the end of the test, a creaming layer is formed. For injection purposes, the floated material is separated from the reaction medium and taken up with 1.5 l of water.

### EXAMPLE 3

### Influence of the Surfactant Concentration on the Particle Properties

(a) Production of the Primary Dispersion

Primary dispersions are produced analogously to Example 1(a) with triton concentrations of respectively 0.1% (0.5 g), 0.5% (2.5 g), 1% (5 g), 2% (10 g), and 10% (50 g).

b) Production of the Microcapsule Suspension

Gas-filled microcapsules are built up from the primary dispersions that are obtained according to (a). Primary dispersions are used with different size distribution with a mean diameter of 50 nm, 100 nm and 250 nm (dynamic light scattering). The process is performed as described under Example 1(b).

(c) Particle Size of the Nanoparticles in Primary Dispersion

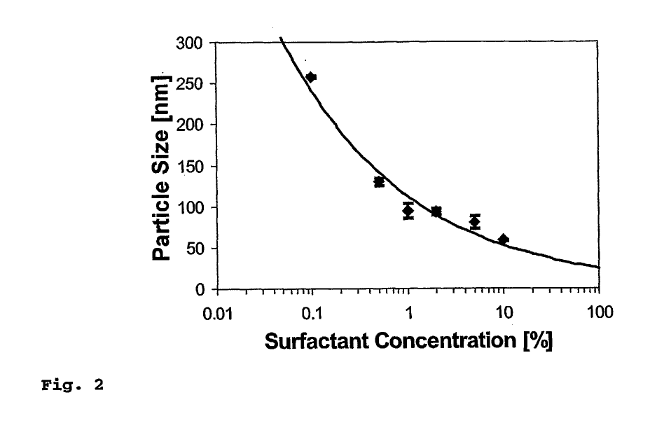

The primary dispersions are characterized by means of dynamic light scattering with respect to the particle size. FIG. 2 shows the measured mean particle diameter (intensity-weighted). The size of the polymerized nanoparticle systematically drops with increasing surfactant concentration.

(d) Particle Size of the Gas-Filled Microcapsules

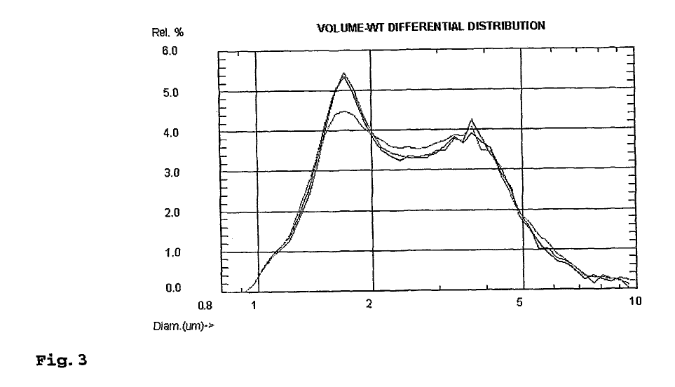

FIG. 3 shows the volume-weighted size distribution (particle counter of the Particle Sizing Systems Company, AccuSizer770 type) of the gas-filled microcapsules, produced according to Example 3(b) in the measuring range of 0.8-10 μm. The particle size distribution of the primary dispersion has no significant influence on the size distribution of the gas-filled microcapsules.

(e) Ultrasound Damping of the Gas-Filled Microcapsules

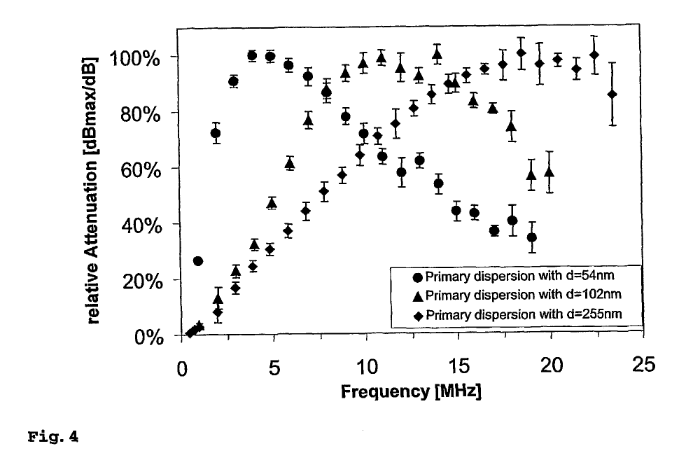

To characterize the properties in the ultrasound field, the frequency-dependent ultrasound absorption (ultrasound damping) of the microcapsules produced according to Example 3 is determined. FIG. 4 shows the absorption spectrum of the gas-filled microcapsules in the ultrasound frequency range of 1 to 25 MHz. It was standardized to the damping maximum. The range of maximum absorption shifts to higher ultrasound frequencies with increasing size of the primary particles used for the production of microcapsules.

(f) Microcapsule Wall Thickness

With identical dispersing conditions, microcapsules of the same particle size (Example 3(d)) but with greatly different properties in the ultrasound field (Example 3(e)) are obtained. If the ultrasound frequency of maximum absorption (Example 3(e)) is considered as a resonance frequency of the microcapsule population, this process can be described with conventional theories on the interaction of ultrasound with gas bubbles (N. de Jong Acoustic Properties of Ultrasound Contrast Agents, Rotterdam, Diss. 1993) and can be attributed to different microcapsule wall thicknesses.

The resonance frequency of gas bubbles (without a shell) in a liquid is inversely proportional to the diameter of the gas bubbles.

$${f}_{o}^{Blase} = \frac{1}{2 π r} \sqrt{\frac{3 γ P}{ρ}}$$

with:

fₒ=resonance frequency [s⁻¹]; r=radius of the bubble [m];

γ=adiabatic exponent of the gas (Cp/Cv; here 1.4); P=prevailing pressure (here 1·10⁵ N/m²); p=density of the liquid (here 1·10³ kg/m³)

According to the above-mentioned theory, this dependence for microcapsules must be expanded by an additive term that contains a shell parameter:

$$\begin{matrix}{f}_{o}^{microcapsule} = \frac{1}{2 π r} \sqrt{\frac{3 γ}{ρ} ( P + π / 3 \frac{{S}_{e}}{γ r}} & ( 2 ) \\ {}^{″} Shell {parameter}^{″} ≡ {S}_{e} \approx \frac{8 π E}{( 1 - v )} ( r - {r}_{i} ) & ( 3 )\end{matrix}$$

E is the modulus of elasticity [N/m²] of the shell material—of the polymer; v is the Poisson ratio (assumes values of 0 to 0.5), which describes the ratio of volume change of an element to expansion, and (r-rᵢ) is the difference between outside and inside radius of the microcapsules—and thus wall thickness [m].

FIG. 3 (Example 3(d)) shows that the size distribution of the microcapsules does not differ when using primary dispersions of different size distribution. FIG. 4 (Example 3(e)) proves that-the resonance frequency of the microcapsules with increasing size of the primary particles used to build up the microcapsules shifts to higher ultrasound frequencies. With the mean size of the microcapsules known from FIG. 3 (diameter about 2.5 μm) and the measured resonance frequency of FIG. 4, the shell parameter can be calculated with above equation (2).

TABLE 1
Comparison of the measurement variables for calculating
the shell parameters according to equation (2)

| Diameter of | | | |
|---|---|---|---|
| the primary | Microcapsule | Resonance | Shell |
| particles | size | frequency | parameter |
| d [nm] | r [μm] | fₒ [MHz] | Sₑ [N/m] |
| 54 (±13) | 1.25 (±0.75) | 4.5 (±1) | 0.3 (±0.1) |
| 102 (±26) | 1.25 (±0.75) | 11 (±1) | 2.8 (±0.7) |
| 255 (±38) | 1.25 (±0.75) | 20 (±2) | 9.7 (±2.4) |

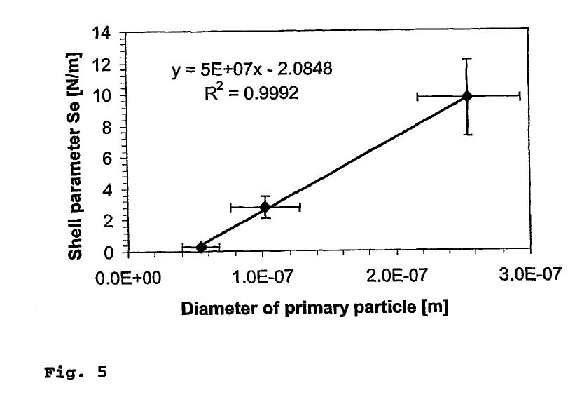

The plotting of shell parameter Sₑ versus the mean diameter of the nanoparticles in the primary dispersion yields a linear dependence (FIG. 5). Obviously, the size of the primary particles directly determines the wall thickness of the microcapsules produced therefrom.

The slope contains both modulus of elasticity E and v (see above: definition of shell parameter equation (3)). Since v can assume only values between 0 and 0.5, a modulus of elasticity of 1-2·10⁶ N/m² can be easily assessed from the slope (5·10⁷ N/m²), which lies between that of high-molecular polyacrylic acid esters (3·10⁹ N/m²) and vulcanized rubber (3-8·10⁵ N/m²).

### EXAMPLE 4

### Functionalized Gas-Filled Microcapsules

Process Variant IV

(a) Production of the Primary Dispersion

For injection purposes, 500 ml of water is loaded into a 1 l glass reactor with a diameter to height ratio of 0.5, and a pH of 2.5 is set by adding IN hydrochloric acid and a reactor temperature of 290.5 K is set. While being stirred with a propeller stirrer, 5.0 g of octoxynol is added and stirred until the octoxynol is completely dissolved. Then, under the same stirring conditions, 7 g of cyanoacrylic acid butyl ester is added in drops over a period of 15 minutes and stirred for another 2 hours.

(b) Production of the Microcapsule Suspension

The primary dispersion is dispersed for two hours with an Ultraturrax (e.g., IKA, T25 type) at high shear gradients (idle speed of the Ultraturrax about 20,500 min⁻¹). By the dispersing, a self-gassing of the process medium is carried out with the result of a strong formation of foam. After the end of the reaction, a creaming layer of gas-filled microcapsules is formed. For injection purposes, the floated material is separated from the reaction medium and taken up with 375 ml of water. The microcapsule suspension that is thus produced has a polymer content of 8.45 mg/ml with a pH of 3.8 (24.1° C.).

(c) Functionalization of gas-filled microcapsules by partial side-chain hydrolysis

While being stirred, 50 ml of the microcapsule suspension according to Example 4(b) is mixed with 100 ml of sodium hydroxide solution of concentrations 6.0·10⁻⁵ mol/l (c1), 6.6·10⁻⁴ mol/l (c2) and 7.2·10⁻³ mol/l (c3). In the reaction batch, pH values of 7.7 (c1), 10.6 (c2) and 11.7 (c3) result. After about 2 hours of reaction time, a pH of 3 is set with hydrochloric acid.

(d) Particle Size of Gas-Filled Microcapsules

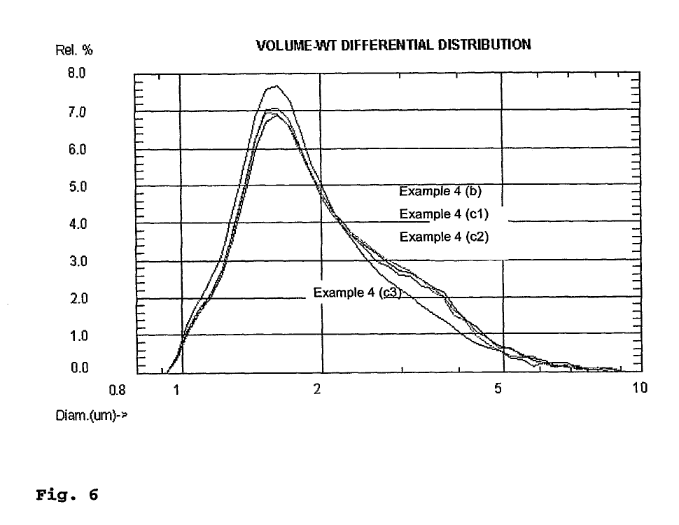

FIG. 6 shows the volume-weighted size distribution (particle counter of the Particle Sizing Systems Company, AccuSizer770 type) of the gas-filled microcapsules in the measuring range of 0.8 to 10 μm. Only at the maximum sodium hydroxide solution concentration (c3) can a slight change of the size distribution be observed. This can be attributed to a reduction of the wall thickness. Under the conditions described here, no aggregation and also no change in particle concentration can be observed.

(e) In-Vitro Ultrasound Effectiveness of Microcapsules According to Example 4

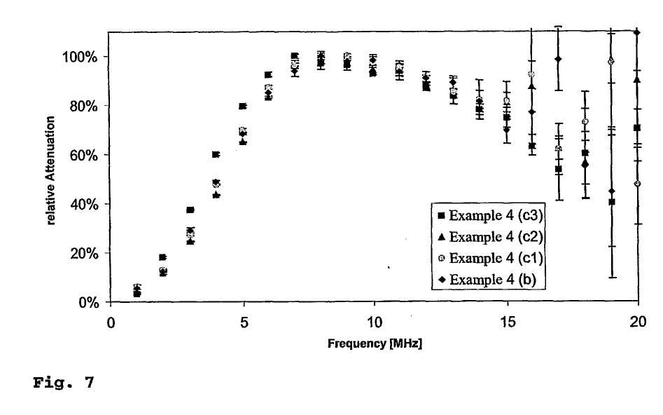

To characterize the microcapsule properties in the ultrasound field, the frequency-dependent ultrasound absorption (ultrasound damping) of the microcapsules is determined. FIG. 7 shows the absorption spectrum of the gas-filled microcapsules in the ultrasound frequency range of 1 to 20 MHz. It was standardized to the damping maximum. The absorption spectrum for microcapsules according to Example 4 (c1) and 4 (c2) easily shifts to lower ultrasound frequencies compared to the untreated microcapsules according to Example 4(b). For microcapsules according to 4 (c3) (the most vigorous surface treatment with sodium hydroxide solution), the range of maximum absorption clearly shifts to lower ultrasound frequencies. This shifting can be attributed to a reduction of wall thickness and corresponds to the results for particle size distribution.

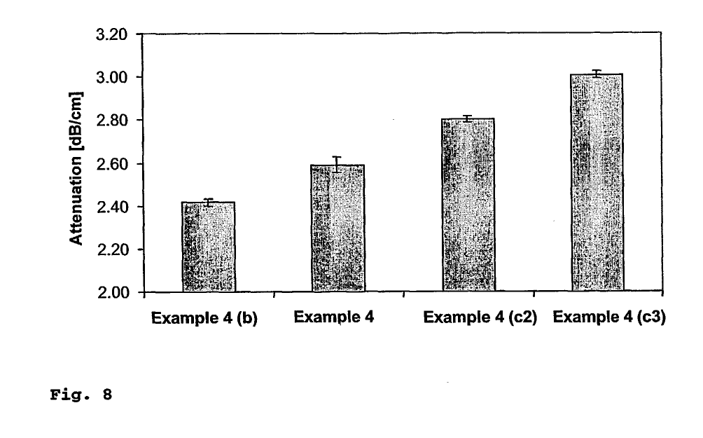

In addition to the relative absorption spectra that are shown in FIG. 7, the absolute values of the ultrasound damping for a diagnostically relevant measuring frequency of 5 MHz are also shown in FIG. 8. This comparison shows that the ultrasound effectiveness parameter grows significantly with an increasing degree of functionalization (concentration of the sodium hydroxide solution).

All measurements were made at a constant microcapsule concentration of 2.5 10⁶ particles/ml in 0.01% TritonX100.

### EXAMPLE 6

### Functionalized Gas-Filled Microcapsules

Process Variant III

(a) Production of the Microcapsule Suspension

7 l of an aqueous 1% octoxynol solution is introduced at a pH of 2.5 into a 20 l reactor and mixed with a rotor-stator mixer at high shear gradient so that a self-gassing with strong formation of foam is carried out. 100 g of cyanoacrylic acid butyl ester is quickly (<1 minute) added and dispersed. It is polymerized for 60 minutes with self-gassing, whereby gas-filled microcapsules form. In a separatory funnel, the floated material is separated, the subnatant is drained off, and the floated material is resuspended with 3 l of an aqueous 0.02% octoxynol solution. The microcapsule suspension that is thus obtained has a polymer content of 9.46 mg/ml, a density of 0.943 g/ml and a pH of 3.5.

b) Functionalization of gas-filled microcapsules by partial side-chain hydrolysis

2418 g (b1) or 2500 g (b2) of a microcapsule suspension according to (a) is mixed with 239 g (b1) or 501 g (b2) of sodium hydroxide solution of concentration 8·10⁻² mol/l while being stirred. pH values of 11.8 (b1) or 12.1 (b2) result in the reaction batch. It is stirred for 20 minutes at room temperature. Then, the pH is set at 3.5 with IN hydrochloric acid.

(c) Particle Size of the Gas-Filled Microcapsules

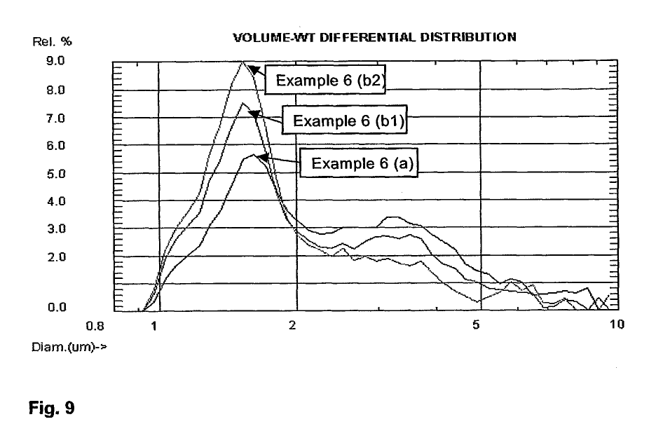

FIG. 9 shows the volume-weighted size distribution (particle counter of the Particle Sizing Systems Company, AccuSizer 770 type) of the gas-filled microcapsules, that are produced in the measuring range of 0.8 to 10 μm.

(d) Freeze-drying and determination of the butanol content

Proof of Functionalization

For injection purposes, the suspensions according to Example 6(a) and (b) are diluted with water to a polymer content of about 4 mg/ml. Then, in each batch, a polyvinylpyrrolidone concentration of 10% is set, the suspensions are formulated up to 10 g and freeze-dried.

By means of gas chromatography (head-space method; carrier gas: helium; stationary phase: DB624; device: Perkin-Elmer HS40), the 1-butanol content is determined. Compared to non-functionalized microcapsules according to Example 6(a), a 5× higher value is found for functionalized microcapsules according to Example 6(b1) and 20 times as much 1-butanol is found according to Example 6(b2).

TABLE 2
Butanol content determinations

| | Ratio of Butanol Content to |
|---|---|
| | PBCA Content |
| Example 6 (a) | 0.32 μg/mg |
| Example 6 (b1) | 1.70 μg/mg |
| Example 6 (b2) | 6.72 μg/mg |

(e) Antagonistic Titration for Surface Charge Determination

Proof of Functionalization

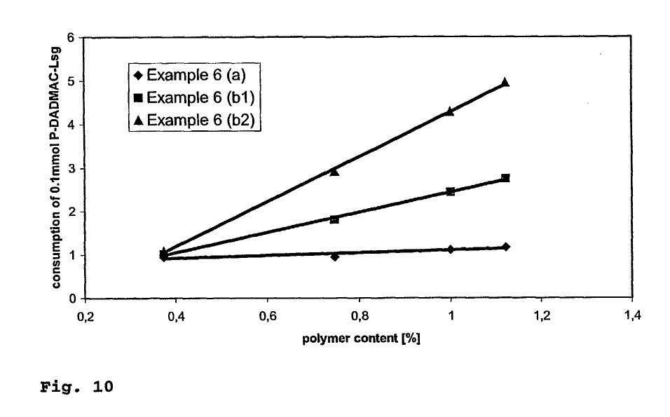

The charge determinations are performed with a Mütek titrator PCD 02. The samples are titrated in four dilutions (0.3%<polymer content<1.2%) up to charge neutrality with P-DADMAC solution of the concentration of 0.1 mmol. The charge density is calculated from the compensating lines of individual measurements (consumption of P-DADMAC solution at a given polymer content) and the average particle radius of the microcapsules. In FIG. 10, the measuring results are depicted. For non-functionalized microcapsule suspensions according to Example 6(a), no significant charge density can be determined with this method. For functionalized microcapsules according to Example 6(b), a surface charge density of 4.2 μC/cm² (b1) or 5.1 μC/cm² (b2) follows from the slope of the compensating lines. The charge density increases with increasing sodium hydroxide solution concentration in the reaction. For non-functionalized microcapsules according to Example 6(a), a compensating line without a significant slope is produced.

(f) Dilution Stability

For injection purposes, the microcapsule concentration of the suspensions according to Examples 6(a) and (b) is set with water at 5·10⁹ particles (≧1 μm) per ml (particle counter of the Particle Sizing Systems Company, AccuSizer 770 type). To study the dilution stability, in each case 1 ml of the suspensions is diluted with isotonic common salt solution of increasing volumes and studied visually for microcapsule aggregates after 30 minutes of service life (while being stirred slightly).

While the non-funtionalized microcapsules already visibly tend toward aggregation after a volume increase by 500% (1 ml of microcapsule suspension+5 ml of isotonic common salt solution), the functionalized microcapsules are still aggregate-free after a volume increase by 2000% (1 ml of microcapsule suspension+20 ml of isotonic common salt solution).

(g) Degradation In-Vitro

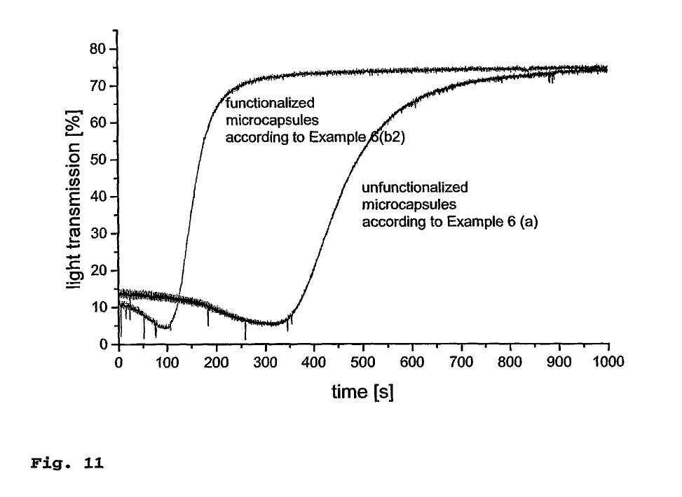

For injection purposes, the microcapsule concentration of the suspensions is set with water at 5·10⁹ particles (≧1 μm) per ml (particle counter of the Particle Sizing Systems Company, AccuSizer 770 type). To study the degradation kinetics, time-dependent measurements of cloudiness are made at a wavelength of 790 nm (spectrometer of the Shimadzu Company UV-2401PC) and 25° C. To this end, 0.5 ml of the respective formulation is diluted directly in the measuring cell with 2.0 ml of sodium hydroxide solution (concentration: 1.25·10⁻³ mol/l), so that a pH of 11 is set. After 60 seconds, the measuring is started. By way of example, FIG. 11 shows the results for gas-filled microcapsules that are produced according to Example 6(a) (non-functionalized) and Example 6(b2) (functionalized).

Compared to the untreated sample, the dwell time of the functionalized microcapsule is reduced by about 75% and the maximum dissolution rate (increase in inflection point) is increased by 0.37% trans./s (non-functionalized) to 0.86% trans./s (functionalized).

(h) Ultrasound Effectiveness In-Vivo

A beagle (about 12 kg of body weight) is anesthetized (inhalational anesthesia air+2-3% enflurane; spontaneous respiration) and prepared for a sonographic study of the heart. The study is done with an ultrasound device of the ATL Company (UM9 type, L10/5 transducer) in the spectral Doppler mode for low, medium and high transmit amplitudes.

In each case, a test animal receives an intravenous administration of the test substance that is produced according to Example 6(a) (non-functionalized) and Example 6(b2) (functionalized).

As a reference substance, a contrast medium is used that was produced analogously to Example 23 of WO 93/25242 with polyvinylpyrrolidone as a cryoprotector.

The dose that is used was 3·10⁷ particles per kg of body weight for all test substances.

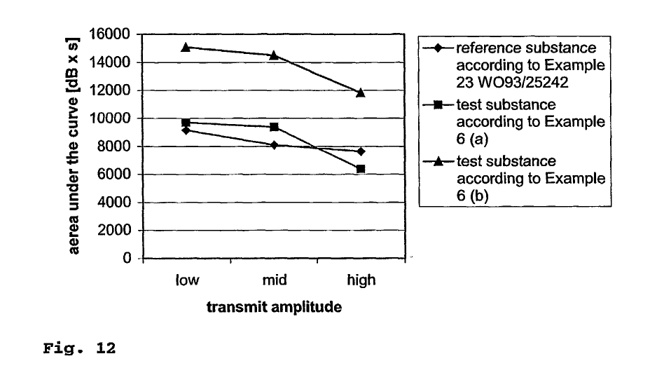

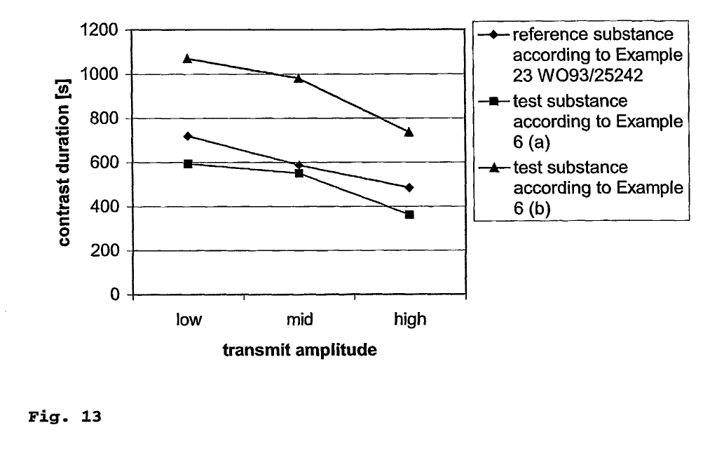

FIG. 12 shows the integral Doppler intensity (surface under the intensity-time curve), and FIG. 13 shows the ultrasound contrast period of the reference substance and test substances.

It is discernible that the functionalized gas-filled microcapsules according to Example 6(b2) have clearly better contrasting properties than the non-functionalized gas-filled microcapsules of the prior art. This is discernible in a higher integral intensity and an extension of the diagnostic time window.

The effectiveness values of the functionalized gas-filled microcapsules according to Example 6(b2) are increased by 50%, and the contrast times are extended by about the factor 2.

### EXAMPLE 7

### Functionalized Gas-Filled Microcapsules

Process Variant I

7 l of an aqueous 1% octoxynol solution with a pH of 2.5 is loaded into a 20 l reactor and dispersed with a rotor-stator mixer at a high shear gradient so that self-gassing with a strong formation of foam is carried out. A mixture of 75 g of cyanoacrylic acid butyl ester and 15 g of cyanoacrylic acid is quickly (<1 minute) added and dispersed. It is polymerized for 60 minutes under self-gassing, whereby gas-filled microcapsules are formed. In a separatory funnel, the floated material is separated, the subnatant is drained off, and the floated material is resuspended with 3 l of an aqueous 0.02% octoxynol solution. The suspension that is thus obtained contains gas-filled microcapsules measuring 0.5 to 10 μm (laser diffractometer of the Malvern Instruments Company, MastersizerS type).

### EXAMPLE 8

### Functionalized Gas-Filled Microcapsules

Process Variant II

(a) Production of the Primary Dispersion

For injection purposes, 500 ml of water is loaded into a 1 l glass reactor with a diameter to height ratio of 0.5, and a pH of 1.5 is set by adding 1N hydrochloric acid and a reactor temperature of 290.5 K is set. While being stirred with a propeller stirrer, 5.0 g of octoxynol is added and stirred until the octoxynol is completely dissolved. Then, under the same stirring conditions over a period of 15 minutes, 6.0 g of cyanoacrylic acid butyl ester together with 1.0 g of cyanoacrylic acid are added in drops and stirred for another 2 hours. The primary dispersion that is obtained is measured by means of dynamic light scattering (device: Nicomp Submicron Particle Sizer) and shows nanoparticles in a range of 50 to 120 nm.

(b) Production of the Microcapsule Suspension

The primary dispersion is dispersed for 2 hours with an Ultraturrax (e.g., IKA, T25 type) at high shear gradients (idle speed of the Ultraturrax about 20,500 min⁻¹). By the dispersing, a self-gassing of the process medium is carried out with the result of a strong formation of foam. After the end of the reaction, a creaming layer of gas-filled microcapsules is formed. For injection purposes, the floated material is separated from the reaction medium and taken up with 375 ml of water. The microcapsule suspension that is thus obtained contains microcapsules in a range of 0.5-10 μm (laser diffractometer of the Malvern Instruments Company, MastersizerS type).

(c) Freeze-Drying

40 g of polyvinylpyrrolidone is dissolved in the batch, the suspension is formulated at 5 g and freeze-dried.

### EXAMPLE 9

### Functionalized Gas-Filled Microcapsules

Process Variant V

(a) Production of the Primary Dispersion:

For injection purposes, 500 ml of water is loaded into a 1 l glass reactor with a diameter to height ratio of 0.5, and a pH of 1.5 is set by adding 1N hydrochloric acid and a reactor temperature of 290.5 K is set. While being stirred with a propeller stirrer, 5.0 g of octoxynol is added and stirred until the octoxynol is completely dissolved. Then, under the same stirring conditions over a period of 15 minutes, 7 g of cyanoacrylic acid butyl ester, added in drops, is stirred for another 2 hours.

(b) Functionalization of the Primary Dispersion

In the primary dispersion, a pH of 11 is set with 165 ml of 0.1N sodium hydroxide solution while being stirred, and it is stirred for 20 minutes at room temperature. Then, a pH of 3 is set with 13 ml of 0.1N hydrochloric acid.

(c) Production of the Microcapsule Suspension

The functionalized primary dispersion is dispersed for 2 hours with an Ultraturrax (e.g., IKA, T25 type) at high shear gradients (idle speed of the Ultraturrax about 20,500 min⁻¹). By the dispersion, a self-gassing of the process medium is carried out with the result of a strong formation of foam. After the end of the reaction, a creaming layer of gas-filled microcapsules is formed.

For injection purposes, the floated material is separated from the reaction medium and taken up with 375 ml of water. The suspension that is thus obtained contains microcapsules in a range of 0.5-10 μm (laser diffractometer of the Malvern Instruments Company, MastersizerS type).

### EXAMPLE 10

### Binding of HSA to Functionalized, Gas-Filled Microcapsules

The microcapsule suspension according to Example 6(b2) is purified by flotation at least 5× from 0.02% Triton-X100 solution. 1 ml of the purified microcapsule suspension with a concentration of 5·10⁹ particles per ml is mixed with 10 μl of a 10% HSA solution and stirred for 60 minutes at 4° C. Then, 10 mg of (1-ethyl-3-(3-dimethylaminopropyl)-carbodiimide hydrochloride (EDC) is added, and the pH is set at 6.5 with 0.1N hydrochloric acid. The incubation is pursued for about 16 hours at 4° C. while being stirred.

The gas-filled microcapsules, to which HSA was bonded, are separated by repeated flotation of unbonded HSA and the by-products. 57% of the amount of protein was bonded to the microcapsules (UV spectroscopy).

### EXAMPLE 11

### Binding of polyethylene glycol to functionalized, gas-filled microcapsules

The microcapsule suspension according to Example 6(b2) is purified by flotation at least 5× from 0.02% Triton-X100 solution. 1 ml of the purified microcapsule suspension with a concentration of 5·10⁹ particles per ml is mixed with 10 μl of a 10% solution of amine-terminated polyethylene glycol (HO—POE—NH₂/3000 Dalton) and stirred for 60 minutes at 4° C. Then, 10 mg of EDC is added, and the pH is set at 6.5 with 0.1N hydrochloric acid. The incubation is pursued for about 16 hours at 4° C. while being stirred. The gas-filled microcapsules, to which HO—POE—NH₂ was bonded, are separated by repeated flotation of unbonded HO—POE—NH₂ and the by-products. 70% of the HO—POE—NH₂ used was bonded to the microcapsules (Colorimetrische Methode mittels Iod-PEG Komplex [Colorimetric Method Using Iodine-PEG Complex], according to G. E. C. Sims, T. J. A. Snope, Ann. Biochem., 107, 60-63 (1980)).

### EXAMPLE 12

### Binding of L-selectin to functionalized, gas-filled microcapsules

The microcapsule suspension according to Example 6(b2) is purified by flotation at least 5× from 0.02% Triton-X100 solution. 1 ml of the purified microcapsule suspension with a concentration of 5·10⁹ particles per ml is rebuffered in 10 mmol of acetate, pH 4.0 and activated with 0.1 M EDC/NHS. Then, it is incubated with 0.25 mg of protein G (5× excess) for one hour at room temperature. The reaction is terminated by a 15-minute incubation with 1 M ethanolamine.

The gas-filled microcapsules, to which protein G was bonded, are purified by repeated washing by means of centrifuging at a maximum of 500 g. The purified, gas-filled protein G-binding microcapsules are incubated overnight with 100 μg of L-selectin-1g-chimera.

50% of the L-selectin amount was bonded to the microcapsules (FACS measurement: saturation series with anti-selectin antibodies).

### EXAMPLE 13

### Binding of streptavidin to functionalized, gas-filled microcapsules with subsequent coupling to biotin-gold particles

The microcapsule suspension according to Example 6(b2) is purified by flotation at least 5× from 0.02% Triton-X100 solution. 1 ml of the purified microcapsule suspension with a concentration of 5·10⁹ particles per ml is mixed with 1 ml of a 2% streptavidin solution and stirred for 60 minutes at 40° C. Then, 10 mg of EDC is added, and the pH is set at 6.5 with 0.1N hydrochloric acid. The incubation is pursued for about 16 hours at 4° C. while being stirred. The gas-filled microcapsules, to which streptavidin was bonded, are separated by repeated flotation from unbonded protein and the by-products.

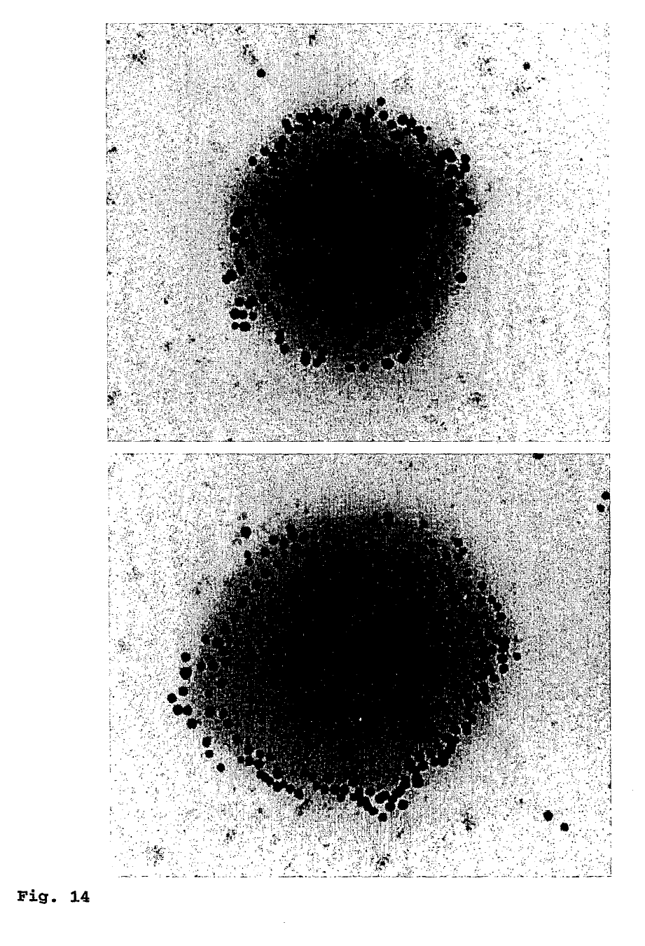

500 μl of the thus purified microcapsule-streptavidin-constructs are mixed at room temperature with 500 μl of a dispersion of biotin-albumin-gold particles (Sigma Biochemicals) with an average diameter of 17-23 nm. The success of coupling is checked by means of electron microscopy (transmission) (FIG. 14).

### EXAMPLE 14

### Nitrogen-filled microcapsules

(a) Production of the Primary Dispersion

For injection purposes, 500 ml of water is loaded in nitrogen countercurrent into a 1 l nitrogen-flushed glass reactor with a diameter to height ratio of 0.5, and a pH of 1.5 is set by adding 1N hydrochloric acid and a reactor temperature of 290.5 K is set. While being stirred with a propeller stirrer, 5.0 g of octoxynol is added and stirred until the octoxynol is completely dissolved. Via a glass tube, nitrogen is directed into the solution for 24 hours.

Then, 7 g of cyanoacrylic acid butyl ester is added in drops into the nitrogen countercurrent under the same stirring conditions over a period of 15 minutes, and it is stirred for another 2 hours.

b) Production of the Microcapsule Suspension

The primary dispersion is dispersed in nitrogen countercurrent for 2 hours with an Ultraturrax (e.g., IKA, T25 type) at high shear gradients (idle speed of the Ultraturrax about 20,500 min⁻¹). By the dispersing, a self-gassing of the process medium is carried out with the result of a strong formation of foam. After the end of the reaction, a creaming layer of gas-filled microcapsules is formed.

The floated material is separated from the reaction medium and taken up with 375 ml of water, which was previously saturated with argon. Then, in argon countercurrent, up to 10 g each is decanted and sealed gastight. The suspension that is thus obtained contains microcapsules in the range of 0.5-10 μm (laser diffractomer of the Malvern Instruments Company, MastersizerS type).

(c) Detection of nitrogen filling

The nitrogen detection is performed with the aid of Raman spectroscopy (device: Dilor Labram) in the gas chamber above the microcapsule suspension directly in the glass vessel. To this end, first a measurement is made in the range of 2200 to 2400 cm⁻¹ and 50 to 150 cm⁻¹ (null value). Then, the microcapsules are destroyed with the aid of ultrasound (30 minute ultrasound bath: device: Bandelin Sonorex) and measured again. After the microcapsules are destroyed, the N₂ vibration band at 2300 cm⁻¹ and the N₂ specific rotation bands at 50 to 150 cm⁻¹ can be seen clearly.

### EXAMPLE 15

### Functionalized, Gas-Filled Microcapsules

Functional monomer glycidylmethacrylate

(a) Production of the Primary Dispersion

For injection purposes, 500 ml of water is loaded into a 1 l glass reactor with a diameter to height ratio of 0.5, and a pH of 1.5 is set by adding 1N hydrochloric acid and a reactor temperature of 290 K is set. While being stirred with a propeller stirrer, 5.0 g of octoxynol is added and stirred until the octoxynol is completely dissolved. 6.0 g of cyanoacrylic acid butyl ester is mixed with 1.0 g of glycidylmethacrylate (2,3-epoxypropylmethacrylate) and in addition 100 mg of AIBN (azo-bis-isobutyronitrile) is dissolved in the mixture under dry nitrogen atmosphere.

Then, the mixture is added in drops into the acidic octoxynol solution over a period of 15 minutes while being stirred with a propeller stirrer—without self-gassing, and it is stirred for another 24 hours at 318 K. The primary dispersion that is obtained is measured by means of dynamic light scattering (device: Nicomp Submicron Particle Sizer) and shows nanoparticles in the range of 30 to 200 nm.

(b) Production of the Microcapsule Suspension

The primary dispersion is dispersed for 2 hours with an Ultraturrax (e.g., IKA, T25 type) at high shear gradients (idle speed of the Ultraturrax about 20,500 min⁻¹). By the dispersing, a self-gassing of the process medium is carried out with the result of a strong formation of foam. After the end of the reaction, a creaming layer of gas-filled microcapsules is formed. For injection purposes, the floated material is separated from the reaction medium and taken up with 375 ml of water. The microcapsule suspension that is thus obtained contains microcapsules in a range of 0.5-10 μm (laser diffractometer of the Malvern Instruments Company, MastersizerS type).

### EXAMPLE 16

### Functionalized, Gas-Filled Microcapsules

Functional monomer 4-aminostyrene

(a) Production of the Primary Dispersion

For injection purposes, 500 ml of water is loaded into a 1 l reactor with a diameter to height ratio of 0.5, and a pH of 1.5 is set by adding 1N hydrochloric acid and a reactor temperature of 283 K is set. While being stirred with a propeller stirrer, 5.0 g of octoxynol is added and stirred until the octoxynol is completely dissolved. 6.0 g of cyanoacrylic acid butyl ester is mixed with 1.0 g of 4-aminostyrene and added in drops into the acidic octoxynol solution over a period of 15 minutes while being stirred with a propeller stirrer—without self-gassing. The reaction mixture is irradiated with a laboratory UV lamp and stirred for another 24 hours at 283 K. The primary dispersion that is obtained is measured by means of dynamic light scattering (device: Nicomp Submicron Particle Sizer) and shows nanoparticles in the range of 50 to 200 nm.

(b) Production of the Microcapsule Suspension

The primary dispersion is dispersed for 2 hours with an Ultraturrax (e.g., IKA, T25 type) at high shear gradients (idle speed of the Ultraturrax about 20,500 min⁻¹). By the dispersing, a self-gassing of the process medium is carried out with the result of a strong formation of foam. After the end of the reaction, a creaming layer of gas-filled microcapsules is formed. For injection purposes, the floated material is separated from the reaction medium and taken up with 375 ml of water. The thus obtained microcapsule suspension contains microcapsules in the range of 0.5-10 μm (laser diffractometer of the Malvern Instruments Company, MastersizerS type).

### EXAMPLE 17

### Functionalized, Gas-Filled Microcapsules

Functional monomer Inisurf polyethylene glycol azo initiator (PEGA200)

(a) Production of the primary dispersion

For injection purposes, 500 ml of water is loaded into a 1 l reactor with a diameter to height ratio of 0.5, and a pH of 1.5 is set by adding 1N hydrochloric acid and a reactor temperature of 290 K is set. While being stirred with a propeller stirrer, 5.0 g of octoxynol is added and stirred until the octoxynol is completely dissolved. 1.0 g of polyethylene glycol azo initiator ([NC(CH₃)₂COO(CH₂CH₂O)₅H]₂) (Tauer, K.; Polym. Adv. Techn. 6, 435 (1995)) is dissolved in 6.0 g of cyanoacrylic acid butyl ester at room temperature.

Then, the mixture is added in drops into the acidic octoxynol solution over a period of 15 minutes while being stirred with a propeller stirrer—without self-gassing, and it is stirred for another 24 hours at 318 K. The primary dispersion that is obtained is measured by means of dynamic light scattering (device: Nicomp Submicron Particle Sizer) and shows nanoparticles in the range of 30 to 200 nm.

(b) Production of the Microcapsule Suspension

The primary dispersion is dispersed for 2 hours with an Ultraturrax (e.g., IKA, T25 type) at high shear gradients (idle speed of the Ultraturrax about 20,500 min⁻¹). By the dispersion, a self-gassing of the process medium is carried out with the result of a strong formation of foam.

After the end of the reaction, a creaming layer of gas-filled microcapsules is formed. For injection purposes, the floated material is separated from the reaction medium and taken up with 375 ml of water. The thus obtained microcapsule suspension contains microcapsules in the range of 0.5-10 μm (laser diffractometer of the Malvern Instruments Company, MastersizerS type).

### EXAMPLE 18

### Binding of the MECA79-Antibody to Functionalized, Gas-Filled Microcapsules

The microcapsule suspension according to Example 6(b2) is purified by flotation at least 5× from 0.02% Triton-X100 solution. 1 ml of the purified microcapsule suspension with a concentration of 5·10⁹ particles per ml is rebuffered in 10 mmol of acetate, pH 4.5, and activated with 0.1 M EDC/NHS. Then, it is incubated with 0.25 mg of streptavidin (5× excess) for one hour at room temperature. The reaction is terminated by a 15-minute incubation with 1 M ethanolamine.

The gas-filled microcapsules, to which streptavidin was bonded, are purified by repeated washing by means of centrifuging at a maximum of 500 g. The purified, gas-filled now biotin-binding microcapsules are incubated for 1 hour with 1 mg of biotinylated MECA79 antibodies and then washed. Control microcapsules were produced analogously with use of the biotinylated isotype-IgM antibodies (Clone R4-22). 50% of the amounts of antibodies used was bonded to the microcapsules (FACS measurement: saturation series with anti-IgM-FITC antibodies).

The MECA79 antibody detects the “peripheral node adressin,” a ligand group that occurs constitutively presented only on the high-endothelial venules of the peripheral and mesenteral lymph nodes.

### EXAMPLE 19

### In-Vivo Detection and Sonographic Detection of the Specific Concentration of MECA79-Antibody-Polymer Microcapsules in Peripheral and Mesenteral Lymph Nodes

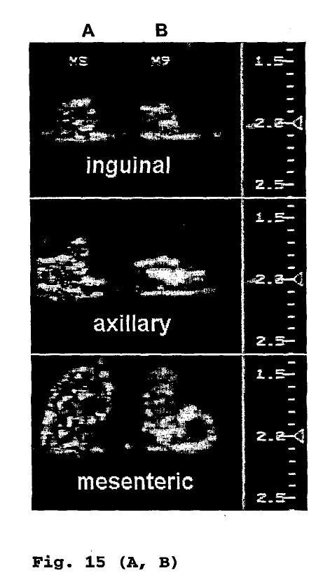

NMRI mice were intravenously injected in isotonic aqueous dispersion with 100 μl of a MECA79-antibody-polymer microcapsule suspension of Example 18 (107 particles per kg of mouse weight). Control mice received comparable amounts of an isotype-IgM-antibody-polymer microcapsule suspension. After 30 minutes, the animals were sacrificed. Peripheral and mesenteral lymph nodes, spleen and kidneys were removed, and a gel bed was embedded as an imaging phantom. The detection of the microcapsules was carried out by scanning the phantom in harmonic color Doppler mode. In the spleen of both animal groups (MECA79 and isotype control), quantitatively comparable signals of microcapsules were found that show that spleen macrophages take up the contrast media in a non-specific manner. In the kidney, no signals of microcapsules were found. In the peripheral and mesenteral lymph nodes, however, signals of microcapsules were found only in the MECA79-antibody animal group (FIG. 15A), but not in the isotype control animal group (FIG. 15B)—a detection for the specific concentration of the MECA79-antibody-microcapsule constructs.

### EXAMPLE 20

### Binding of Anti-Mouse-CD105-Antibodies to Functionalized Gas-Filled Microcapsules

Anti-mouse-CD105-antibodies were bonded analogously to Example 18 to functionalized gas-filled microcapsules.

### EXAMPLE 21

### In-Vivo Detection and Sonographic Detection of the Specific Concentration of Anti-Mouse-CD105-Antibody-Polymer Microcapsules in Tumors

Anti-mouse-CD105-antibody-polymer microcapsule suspensions according to Example 20 were studied in the F9-tumor model in hairless mice. The test substance in non-anesthetized state was administered intravenously as a one-time injection at a dose of 2.1×10⁷ particles per kg of body weight to two tumor-carrying hairless mice. Two control mice received the microcapsule-streptavidin-construct according to Example 13 at the same dosage. After 30 minutes, the animals were sacrificed. The tumors were removed and studied sonographically ex vivo in a water tank with an ultrasound device of the ATL Company (UM9 type, L10-5 transducer) in harmonic color Doppler with use of a high sonic amplitude.

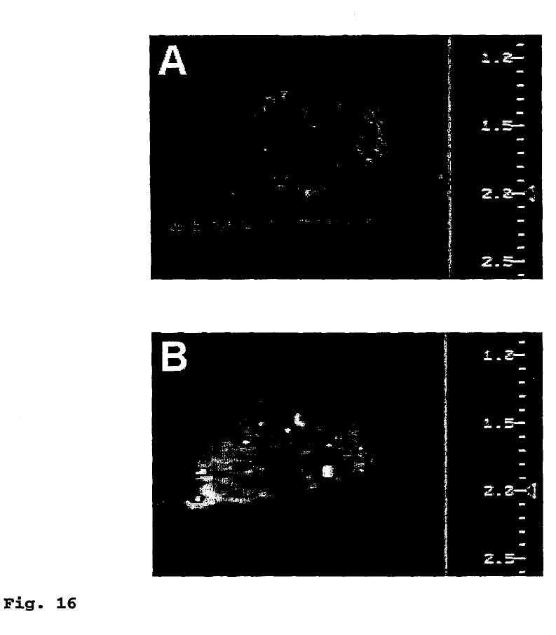

FIG. 16B shows a color coding in the tumor of a mouse that starts from irradiated gas-filled microcapsules according to Example 20. FIG. 16A is free of color signals that are induced by microparticles and shows the control substance. This is a detection of a specific concentration of anti-CD105-antibody-polymer microcapsule constructs in the tumor.

### EXAMPLE 22

### Binding of Anti-Mouse-ICAM-1-Antibodies to Functionalized, Gas-Filled Microcapsules

Anti-mouse-ICAM-1-antibodies were bonded to functionalized gas-filled microcapsules analogously to Example 18. Control microcapsules were produced analogously with use of the biotinylated isotype-IgG-antibody.

### EXAMPLE 23

### In-Vivo Detection and Sonographic Detection of the Specific Concentration of Anti-Mouse-ICAM-1-Antibody-Polymer Microcapsules in the Brain and the Spinal Cord

Anti-mouse-ICAM1-antibody polymer microcapsule suspensions according to Example 22 were studied in the experimentally autoimmune encephalomyelitis model (EAE) of the mouse. The test substance in the non-anesthetized state was administered intravenously as a one-time injection at a dose of 1×10⁹ particles per kg of body weight to two mice. Two control mice received comparable amounts of an isotype-IgG-antibody-polymer microcapsule suspension.

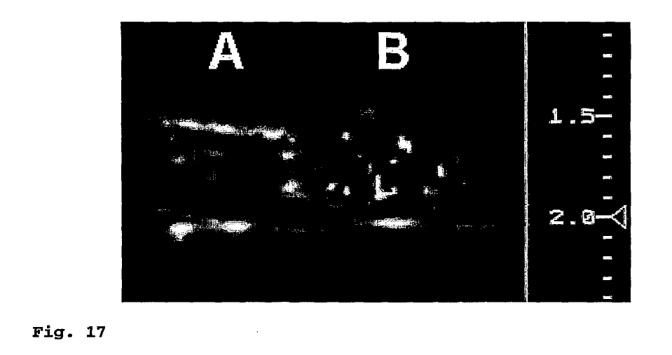

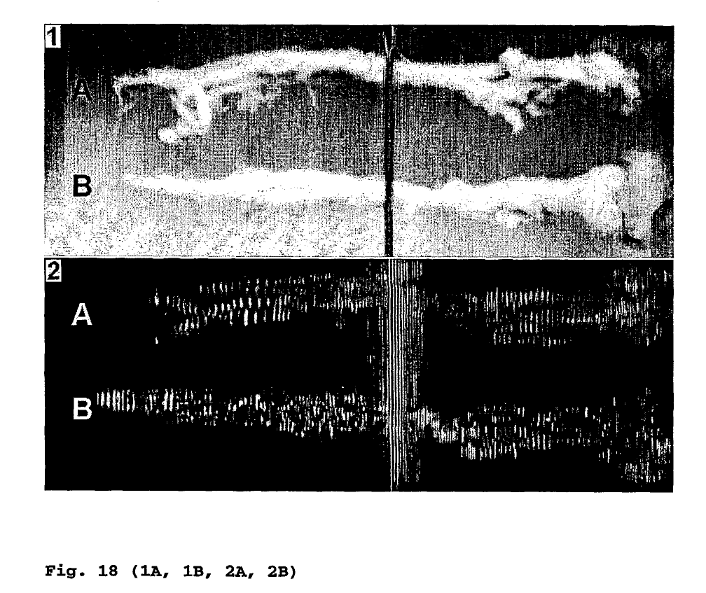

After 4 hours, the animals were sacrificed. Brains and spinal cords were removed and studied sonographically ex vivo in a water tank with an ultrasound device of the ATL Company (UM9 type, L10-5 transducer) in the harmonic color Doppler with use of a high sonic amplitude. FIG. 17B and FIG. 18, 2B show a color coding in the brain and spinal cord/cerebellum of an EAE mouse that starts from irradiated, gas-filled microparticles according to Example 22. FIG. 17A and FIG. 18, 2A are free of color signals that are induced by microparticles and show the control substance.

(FIG. 18, 2: synthesized image of cross sectional images of the spinal cord/cerebellum scanned; FIG. 18, 1: macroscopically anatomical image of the spinal cord/cerebellum).

This is a detection of the specific concentration of the anti-mouse-ICAM1-antibody-polymer microcapsule constructs in the brain and spinal cord.

1. Gas-filled microcapsules, characterized in that the latter contain functionalized polyalkylcyanoacrylate.

2. Gas-filled microcapsules according to claim 1, wherein the functionalized polyalkylcyanoacrylate is produced by copolymerization of one or more alkylcyanoacrylates with a functional monomer.

3. Gas-filled microcapsules according to claim 2, wherein used as a functional monomer is:
cyanoacrylic acid (H₂C═C(CN)—CO—OH), methacrylic acid (H₂C═C(CH₃)—CO—OH), methylenemalonic acid (H₂C═C(CO—OH)₂) and/or α-cyanosorbic acid (H₃C—CH═CH—CH═C(CN)—CO—OH) and/or their derivatives with the general formulas:
H₂C═C(CN)—CO—X—Z (cyanoacrylic acid derivatives),
H₂C═C(CH₃)—CO—X—Z (methacrylic acid derivatives),
H₂C═C(CO—X′—Z′)₂ (methylenemalonic acid derivatives) and
H₃C—CH═CH—CH═C(CN)—CO—X—Z (α-cyanosorbic acid derivatives) with
X=—O—, —NH— or —NR¹— and
Z=—H, —R²—NH₂, —R²—NH—R¹, —R²—SH, R²—OH, R²—HC(NH₂)—R¹ 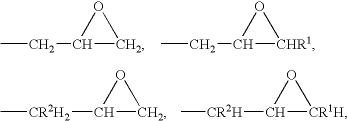
whereby R¹=linear or branched alkyl radical and R²=linear or branched alkylene radical with respectively 1 up to 20 carbon atoms, and whereby both X′ and Z′, in each case independently of one another, have the meaning that is indicated for X and Z,
substituted styrenes (Y—C₆H₄—CH═CH₂) or methylstyrenes (Y—C₆H₄—C(CH₃)═CH₂) with
Y=—NH₂, —NR¹H, —OH, —SH, —R²—NH₂, —R²—NH—R¹, —R²—SH, R²—OH, —R²—HC(NH₂)—R¹
whereby R¹=linear or branched alkyl radical and R²=linear or branched alkylene radical with respectively 1 to up to 20 carbon atoms or polymerizable emulsifiers (Surfmer), initiators with functionality (Inisurf) and chain-transfer agents with functionality (Transsurf).

4. Gas-filled microcapsules according to claim 3, wherein as a functional monomer, cyanoacrylic acid (H₂C═C(CN)—CO—OH) or glycidyl methacrylate ( 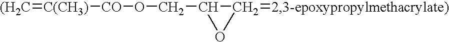
3-epoxypropylmethacrylate) is used.

5. Gas-filled microcapsules according to claim 2, wherein butyl, ethyl and/or isopropylcyanoacrylate is used as an alkylcyanoacrylate.

6. Gas-filled microcapsules according to claim 1, wherein the functionalized polyalkylcyanoacrylate is produced by partial side-chain hydrolysis of a polyalkylcyanoacrylate.

7. Gas-filled microcapsules according to claim 6, wherein butyl, ethyl and/or isopropyl cyanoacrylate is used for the production of polyalkylcyanoacrylate.

8. Process for the production of gas-filled microcapsules according to claims 1 to 5, wherein the following process steps are performed:
(a) Mixing of the functional monomer with one or more alkylcyanoacrylates,
(b) In-situ copolymerization and build-up of microcapsules in acidic, aqueous solution under dispersing conditions in a process step.

9. Process for the production of gas-filled microcapsules according to claims 1 to 5, wherein the following process steps are performed:
(a) Mixing of the functional monomer with one or more alkylcyanoacrylates,
(b) In-situ copolymerization in acidic, aqueous solution under stirring conditions, and
(c) Build-up of microcapsules under dispersing conditions separately from the copolymerization.

10. Process for the production of gas-filled microcapsules according to claims 1, 6 or 7, wherein the following process steps are performed:
(a) In-situ polymerization of one or more alkylcyanoacrylates and build-up of microcapsules in acidic, aqueous solution under dispersing conditions in a process step,
(b) Implementation of partial side-chain hydrolysis by adding lye,
(c) Stopping of the reaction by the addition of acid.

11. Process for the production of gas-filled microcapsules according to claims 1, 6 or 7, wherein the following process steps are performed:
(a) In-situ polymerization of one or more alkylcyanoacrylates in acidic, aqueous solution under stirring conditions,
(b) Build-up of microcapsules under dispersing conditions separately from the copolymerization,
(c) Implementation of partial side-chain hydrolysis by adding lye,
(d) Stopping the reaction by the addition of acid.

12. Process for the production of gas-filled microcapsules according to claims 1, 6 or 7, wherein the following process steps are performed:
(a) In-situ polymerization of one or more alkylcyanoacrylates in acidic, aqueous solution under stirring conditions,
(b) Implementation of partial side-chain hydrolysis by adding lye in primary dispersion,
(c) Stopping the reaction by the addition of acid,
(d) Build-up of microcapsules under dispersing conditions optionally with renewed addition of one or more alkylcyanoacrylates.

13. Process for the production of gas-filled microcapsules according to one of claims 8 to 12, wherein the following process steps are optionally performed:
(a) After the build-up of microcapsules has taken place, one or more flotations with subsequent uptake of the floated material in a physiologically compatible medium,
(b) Even in the case of functionalization by copolymerization with a functional monomer that has already been performed, an additional functionalization by partial side-chain hydrolysis by adding lye and stopping the reaction by the addition of acid,
(c) Filtration, ultrafiltration and/or centrifuging for purification.

14. Process according to one of claims 10 to 13, wherein the partial side-chain hydrolysis is performed at pH values of between 9 and 14 and a reaction time of between 15 minutes and 5 hours.

15. Process according to one of claims 10 to 14, wherein the partial side-chain hydrolysis is stopped in that a pH under 7 is set by the addition of acid.

16. Process according to one of claims 1 to 15, wherein the monomer or monomers are added to acidic, aqueous solution at a concentration of 0.1 to 60%, preferably 0.1 to 10%.

17. Process according to one of claims 1 to 16, wherein one or more of the following surfactants are used:
Alkylarylpoly(oxyethylene)sulfate alkali salts, dextrans, poly(oxyethylenes), poly(oxypropylene)-poly(oxyethylene)-block polymers, ethoxylated fatty alcohols (cetomacrogols), ethoxylated fatty acids, alkylphenolpoly(oxyethylenes), copolymers of alkylphenolpoly(oxyethylene)(s) and aldehydes, partial fatty acid esters of sorbitan, partial fatty acid esters of poly(oxyethylene)sorbitan, fatty acid esters of poly(oxyethylene), fatty alcohol ethers of poly(oxyethylene), fatty acid esters of saccharose or macrogolglycerol ester, polyvinyl alcohols, poly(oxyethylene)hydroxy fatty acid esters, macrogols of multivalent alcohols, partial fatty acid esters.

18. Process according to one of claims 1 to 17, wherein one or more of the following surfactants are used:
ethoxylated nonylphenols, ethoxylated octylphenols, copolymers of aldehydes and octylphenolpoly(oxyethylene), ethoxylated glycerol-partial fatty acid esters, ethoxylated hydrogenated castor oil, poly(oxyethylene)-hydroxystearate, poly(oxypropylene)-poly(oxyethylene)-block polymers with a molar mass<20,000.

19. Process according to one of claims 1 to 18, wherein one or more of the following surfactants are used:
Para-octylphenol-poly-(oxyethylene) with 9-10 ethoxy groups on average (=octoxynol 9,10), para-nonylphenol-poly(oxyethylene) with 30/40 ethoxy groups on average (=e.g., Emulan® 30/Emulan® 40), para-nonylphenol-poly(oxyethylene)-sulfate-Na salt with 28 ethoxy groups on average (=e.g., Disponil® AES), poly(oxyethylene)glycerol monostearate (e.g., Tagat® S), polyvinyl alcohol with a degree of polymerization of 600-700 and a degree of hydrolysis of 85%-90% (=e.g., Mowiol® 4-88), poly(oxyethylene)-660-hydroxystearic acid ester (=e.g., Solutol® HS 15), copolymer of formaldehyde and para-octylphenolpoly(oxyethylene) (=e.g., Triton® WR 1339), polyoxypropylene-polyoxyethylene-block polymers with a molar mass of about 12,000 and a polyoxyethylene proportion of about 70% (=e.g., Lutol® F127), ethoxylated cetylstearyl alcohol (=e.g., Cremophor® A25), ethoxylated castor oil (=e.g., Cremophor® EL).

20. Process according to one of claims 1 to 19, wherein the surfactant or the surfactants are used at a concentration of 0.1 to 10%.

21. Process according to one of claims 1 to 20, wherein the following acids are used: hydrochloric acid, phosphoric acid and/or sulfuric acid.

22. Process according to one of claims 1 to 21, wherein the polymerization and the build-up of microcapsules are carried out at temperatures of −10° C. up to 60° C., preferably in the range between 0° C. and 50° C., especially preferably between 5° C. and 35° C.

23. Process according to one of claims 1 to 22, wherein the period of polymerization and the build-up of microcapsules is between 2 minutes and 2 hours.

24. Process according to one of claims 1 to 23, wherein the gas-filled microcapsules are separated from the reaction medium by flotation, taken up in a physiologically compatible medium and are optionally freeze-dried after a cryoprotector is added.

25. Process according to claim 24, wherein water or physiological common salt solution is used to take up the floated material.

26. Process according to claim 24, wherein as a cryoprotector, polyvinylpyrrolidone, polyvinyl alcohol, gelatin and/or human serum albumin is used.

27. Gas-filled microcapsules, wherein they can be obtained according to the process of one of claims 8 to 26.

28. Gas-filled microcapsules according to one of claims 1 to 7 or according to claim 27, wherein the latter contain specifically binding molecules or the substances that influence kinetics.

29. Gas-filled microcapsules according to one of claims 1 to 7 or according to claim 27, wherein the latter are coupled with specifically binding molecules or the substances that influence kinetics.

30. Gas-filled microcapsules according to claim 28 or 29, wherein the latter are used as specifically binding molecules, antibodies, preferably anti-EDB-FN-antibodies, anti-endostatin antibodies, anti-CollXVIII antibodies, anti-CM201 antibodies, anti-L-selectin-ligand antibodies, such as anti-PNAd antibodies (MECA79 antibodies), anti-CD105 antibodies, anti-ICAM1 antibodies or endogenic ligands, preferably L-selectin and especially preferably chimera L-selectin.

31. Gas-filled microcapsules according to claim 28 or 29, wherein as substances that influence kinetics, synthetic polymers, preferably polyethylene glycol (PEG), proteins, preferably human serum albumin and/or saccharides, preferably dextran, are contained or are coupled to the latter.

32. Gas-filled microcapsules according to claims 29 to 31, wherein the specifically binding molecules or the substances that influence kinetics are coupled directly to the functional groups of the functionalized polyalkylcyanoacrylates.

33. Gas-filled microcapsules according to claims 29 to 31, wherein the specifically binding molecules or the substances that influence kinetics are coupled via a spacer, for example protein G, to the functional groups of the functionalized polyalkylcyanoacrylate.

34. Gas-filled microcapsules according to claims 29 to 31, wherein the specifically binding molecules or the substances that influence kinetics are biotinylated via a streptavidin-biotin coupling to the functional groups of the functionalized polyalkylcyanoacrylate.

35. Gas-filled microcapsules according to claims 28 to 34, wherein the functional groups of the functionalized polyalkylcyanoacrylate are activated.

36. Gas-filled microcapsules according to claims 28 to 35, wherein the functional groups of the functionalized polyalkylcyanoacrylate are activated by EDC (1-ethyl-3-(3-dimethylaminopropyl)-carbodiimide hydrochloride).

37. Use of the gas-filled microcapsules according to claims 1 to 7 and 27 to 36 for ultrasound diagnosis.
